(19) United States
(12) Patent Application Publication
(10) Pub. No.: US 2002/0146723 A1
(43) Pub. Date: Oct. 10, 2002

(54) Candidate region mismatch scanning for genotyping and mutation detection

(76) Inventors: Theodore G. Krontiris, Pasadena, CA (US); Martin Beaulieu, San Diego, CA (US); Steven D. Flanagan, Claremont, CA (US)

Correspondence Address: ROTHWELL, FIGG, ERNST & MANBECK, P.C., 1425 K STREET, N.W., SUITE 800, WASHINGTON, DC, 20005, US

(21) Appl. No.: 09/983,758

(22) Filed: Oct. 25, 2001

Related U.S. Application Data
(60) Provisional application No. 60/242,725, filed on Oct. 25, 2000.

(51) Int. Cl. C12Q 1/68; C12P 19/34

(52) U.S. Cl. 435/6; 435/91.2

(57) ABSTRACT

Use of the E. coli mismatch detection system, MutS, MutL and MutH, in PCR-based, automated, high-throughput genotyping and mutation detection of genomic DNA. Optimal sensitivity and signal-to-noise ratios are dependent upon monovalent cation concentration and MutL concentration. Strategies that can be easily adapted to automation for limiting the analysis to intersample heteroduplexes have been developed.

### CROSS-REFERENCE TO RELATED APPLICATIONS

Benefit is claimed of provisional application No. 60/242,725, filed Oct. 25, 2000.

### STATEMENT REGARDING FEDERALLY SPONSORED RESEARCH

This invention was made in part with government support in the form of grants no. CA 45052 and AG 15720 from the United States Public Health Service. The government may have certain rights in the invention.

### FIELD OF THE INVENTION

This is an invention in the field of DNA mutation detection and genotyping.

### BACKGROUND OF THE INVENTION

DNA sequence variation is both a source of human disease and a means by which disease mechanisms may be elucidated. Linkage analysis, which compares variation among affected relatives, and association tests, which compare variation among affected individuals and controls, are the two major approaches to identifying genes and chromosomal regions affecting human disease susceptibility. Each of these approaches primarily relies on scoring DNA sequence variation in the form of short tandem repeat polymorphisms (primarily microsatellites) or single nucleotide polymorphisms (SNPs). As human genetic research progresses toward a more comprehensive analysis of complex genetic disorders, the number (density) of such markers and the effectiveness with which they are scored in individuals must increase dramatically.

Linkage analysis of adult genetic disorders by genotyping microsatellites often suffers from incomplete information, requiring identity-by-state (ibs) rather than identity-by-descent (ibd) analysis. While this may be largely overcome by using more markers in the regions of interest, this reduces the efficiency—especially if the analysis mandates examination of particular candidate gene regions for which marker occurrence is infrequent and/or uninformative. The need for manual interpretation and human error-checking of genotyping data is also time-consuming, affecting the throughput considerably. A more recent technical approach has been the typing of multiple SNPs. However, the strategies now employed using these markers require an exact knowledge of SNP sequence attributes and location. Since the number of SNPs required for proposed susceptibility studies can be quite large (Halushka et al., 1999; Cargill et al., 1999), the typing of these markers for a sufficiently robust analysis by currently available methods is expensive and often beyond the reach of a typical academic laboratory. Currently available commercial software packages and substantial literature on the subject provide only partial solutions to overcoming the problems inherent to conventional genotyping methodologies.

Within the past few years, various techniques have been developed to detect or score sequence variation, particularly in PCR products. These methods can be divided into two categories: (A) Those detecting unknown sequence variants, including chemical mismatch cleavage (CMC; (Lambrinakos et al., 1999; Ellis et al., 1998; Cotton et al., 1988)), denaturing gradient gel electrophoresis (DGGE; (Myers et al., 1987)), single-stranded conformation polymorphism (SSCP; (Orita et al., 1989)), Detection of Virtually all Mutations-SSCP (DOVAM-S; (Liu et al., 1999)) and others; and (B) those scoring known sequence variants, such as TaqMan™ (Heid et al. 1996), molecular beacon hybridization (Tyagi et al., 1999), Invader™ (Cheung et al., 1998), allele-specific PCR (Ugozzoli et al., 1992), and others. (For reviews, see Glavac et al., 1995; Dianzani et al., 1999; Cotton, 1999; Taylor et al., 1999). None of these strategies performs equally well for scoring genotypes and for mutation detection. To perform both tasks, a technique should be sensitive enough to detect virtually all mutation types and quantitative enough that data may be translated into allele sharing status. Moreover, for high-throughput purposes, the method should be easy to optimize for automated typing of many loci.

Genomic mismatch scanning (GMS) is a hybridization-based technique designed to enrich ibd regions between two individuals without the need for genotyping or sequencing (Nelson et al., 1993). In other words, genetic variation may be exploited without the effort and expense of characterizing it carefully. Regions of ibd, once selected by GMS, can then be used for mapping by hybridization to a microarray containing ordered clones of genomic DNA (Nelson et al., 1993; McAllister et al., 1998; Cheung et al., 1998; Cheung et al., 1998; Nelson, 1995; Welford et al., 1998). GMS employs the E. coli mismatch repair enzymes MutH, MutL and MutS (Lahue et al., 1989) to identify DNA regions that contain mismatches in DNA fragments from different sources (cases, relatives, controls, etc.). MutS has increased binding affinity for single-base mismatches and one to four nucleotide insertions or deletions (Parker et al., 1992). Only C-C mismatches are weakly recognized. Following MutS binding to heteroduplex DNA, MutL is recruited and activated. In the presence of ATP, the complex then binds and activates MutH, a latent endonuclease that cleaves DNA 5′ to a nearby d(GATC) site. The mismatch and the cleavage sites may be separated by as much as 1 kb (Yamaguchi et al., 1998; Dao et al., 1998).

Two previous studies have shown that mismatch scanning using bacterial Mut enzymes could be used for mutation detection on PCR products (Smith et al., 1996; Lishanski et al., 1994). However, strategies are needed for performing genotyping by mismatch scanning on PCR amplified candidate regions. Such strategies and experimental conditions useful for performing genotyping, including quantitative genotyping, have not yet been elaborated.

The present invention satisfies the above need in the art, providing strategies, along with experimental conditions, for performing genotyping and mutation detection in candidate regions of DNA by mismatch scanning. In particular, several strategies have been developed that can be easily adapted to automation for limiting the analysis to intersample heteroduplexes. Thus, the principle barriers to using this methodology, which is herein designated PCR Candidate Region Mismatch Scanning (PCR-CRMS), in cost-effective, high- throughput settings have been removed.

The publications and other materials used herein to illuminate the background of the invention, and in particular cases, to provide additional details respecting the practice of the invention, are incorporated herein by reference

### SUMMARY OF THE INVENTION

The present invention involves candidate region mismatch scanning for genotyping or mutation detection in a sample. The method includes amplifying a candidate region of DNA, denaturing and reannealing the amplified DNA, and then digesting the reannealed DNA in the presence of a mismatch detection system to cleave mismatch-containing DNA at the candidate region. The DNA cleaved may then be determined. The preferred mismatch detection system is the E. coli mismatch detection system using MutHLS enzymes.

In one embodiment, a method of genotyping or detecting a mutation in a DNA sample by candidate region mismatch scanning comprises amplifying a candidate region of the DNA that includes at least one 5′ GATC 3′ site, denaturing and reannealing the amplified DNA, digesting the reannealed DNA with the E. coli mismatch detection enzymes, MutS, MutL and MutH, to cleave mismatch-containing DNA at the 5′ GATC 3′ site, and determining the fraction of DNA cleaved.

In another embodiment, a method of genotyping a DNA sample by candidate region mismatch scanning comprises amplifying a candidate region of the DNA that includes at least one 5′ GATC 3′ site, and mixing the amplified DNA with a detectably-labeled probe, preferably prepared by amplifying the corresponding region of a homozygous reference sample. The amplified DNA is then denatured and reannealed in the presence of the probe to produce unlabeled homoduplexes and labeled heteroduplexes, followed by digesting the reannealed DNA with the E. coli mismatch detection enzymes, MutS, MutL and MutH, to cleave mismatch-containing DNA at the 5′ GATC 3′ site. The fraction digested of the single stranded labeled probe is then determined.

An alternative embodiment involves a method of determining allele-sharing status between sibs by candidate region mismatch scanning. The method comprises separately amplifying corresponding candidate regions of genomic DNA samples from a sib pair, which candidate regions contain at least one 5′ GATC 3′ site. The method then involves labeling one amplified DNA with a detectable label, and mixing the unlabeled and labeled amplified DNA's, with the unlabeled DNA present in sufficient excess to maintain the quantitative aspects of the method. The mixed amplified DNA's are then denatured and reannealed to produce labeled homoduplexes and labeled heteroduplexes, followed by digesting the reannealed DNA with the E. coli mismatch detection enzymes, MutS, MutL, and MutH, to cleave mismatch-containing DNA at the 5′ GATC 3 site. The fraction of the labeled DNA cleaved is then determined.

In a preferred embodiment, amplification is carried out by polymerase chain reaction (PCR) using a high-fidelity DNA polymerase.

### BRIEF DESCRIPTION OF THE FIGURES

FIG. 1 is a schematic representation of target PCR products used to optimize PCR-CRMS. PCR primers were designed to amplify DNA fragments from exon 3 of the human CDKN1A gene. Known polymorphisms are designated by solid arrowheads. All the targets were PCR-amplified using the same forward primer (→). Three different lengths of target DNA—260, 516 and 969 bp—were amplified using specific reverse primers (→) Therefore, all targets carry the same mutations, along with the same dam reporter site (GATC; vertical bars). The dam sites are 95 bp from the end of each amplicon and 45 bp away from the first mismatch. The two polymorphic sites are 13 bp apart.

FIG. 2 is a schematic representation and gel photograph illustrating PCR-CRMS genotyping with self-annealed PCR products. A PCR product, either heterozygous (left, top) or homozygous (right, top), is heat-denatured and reannealed to self. The heterozygous sample is expected to generate equal amounts of homoduplex (perfectly matched; PM) and heteroduplex (mismatched; MM). However, the homozygous sample generates only one homoduplex (PM) molecules. MM duplexes are specific targets for activated MutH. Following MutHLS treatment, 50% of the heterozygous sample was digested; only a 5% background level of cleavage was observed with the homozygous sample.

FIGS. 3A and 3B are gel photographs illustrating optimization of PCR-CRMS with longer targets amplified from the CDKN1A locus. The assay conditions were further optimized to accommodate the 516 bp (A) and 969 bp (B) PCR products. Optimal conditions were predicted using the Taguchi method. Homozygous samples (PM), as well as heterozygous samples (MM), were used as negative and positive controls, respectively. Product digestion is quantitated as the percentage of the fragment cleaved relative to input. MM:PM, a measure of signal to noise, represents the ratio of the heterozygous fraction digested to the homozygous fraction digested. Also shown are the final concentrations of DMSO (%) and KCl (mM) added to the reaction.

FIGS. 4A and 4B are gel photographs illustrating effects of polymerase type and fluorescence-tagged primers on PCR-CRMS signal-to-noise ratio. Either AmpliTaq Gold™ or Expand™ enzyme was used to PCR-amplify target DNA's. For each enzyme, either an unlabeled or FAM-labeled forward primer was used for PCR amplification. As in FIG. 2, percent cleavage and MM:PM ratio are given below each reaction set.

FIGS. 5A-C are graphs illustrating the effect of potassium chloride concentration on PCR-CRMS. A) Potassium chloride titration performed on the 260-bp PCR product as target. Homozygous samples (forward arrowhead, PM) heterozygous sample (backward arrowhead; MM). B) Correlation of KCl concentration with length of target DNA. PCR-CRMS was performed on homozygous amplicons of the indicated size from different loci. Nonspecific cleavage at various KCl concentrations is given on the vertical axis. C) Near linear correlation of KCl for optimal cleavage rate ratios with the length of eight different target DNA's. The correlation coefficient (r²) and linear regression equation are shown.

FIG. 6 is a gel photograph illustrating the effect of GATC site position on PRR-CRMS efficiency. Upper, schematic representation of target fragments with distance between the GATC site and the end of the DNA fragment at 95 bp and 64 bp, respectively (solid bar). Lower, electrophoresis and quantitation of PCR-CRMS products. Relative activity is the ratio of MM cleavage for the two targets.

FIG. 7 is a schematic representation of PCR-CRMS strategy employing a single-strand reference probe (ssf probe). A target locus of interest is PCR amplified with standard primers A reference probe is also amplified using a fluorescence-labeled forward primer and a biotin-tagged reverse primer Following purification of single-strand, labeled reference probe by streptavidin binding, the ssf probe is mixed with the test sample PCR products in a ratio of 1:5 to 1:10. The solution is heat-denatured and reannealed in assay buffer. The fluorescent heteroduplex thus formed are the targets of PCR-CRMS and are the only duplexes detected using the ABI 377™ automatic sequencer. The ssf probe is forced to hybridize with the minus strand of the unknown sample forming heteroduplexes. Quantitative analysis of the electropherogram (GeneScan™) provides the extent of mismatch-directed cleavage; in genotyping mode, this corresponds to allele-sharing status. The circles on the left side of the duplex represent a fluorescent label, while the single circle on the right side of the duplex represents a biotin tag.

FIGS. 8A and 8B are, respectively, electropherograms and a graph illustrating PCR-CRMS assay adapted to an automatic sequencer. A) Three electropherograms representing of the ratios of PCR-CRMS products obtained with a wild-type ssf probe and three types of genotypes. Upper: Homozygous wild-type DNA (wt/wt); middle: heterozygous wild-type/variant DNA (wt/variant); lower: homozygous variant DNA (variant/variant). B) Frequency distribution of the % digestion data obtained from a blinded PCR-CRMS assay performed on 58 previously genotyped DNA's. The ssf probe was wild-type. The open and solid bars represent samples previously genotyped as homozygotes and heterozygotes, respectively.

FIGS. 9A and 9B are electropherograms illustrating direct comparison of two sample DNA's. A 969 bp target region from CDKN1A (see FIG. 1) was PCR-amplified from DNA of several individuals with known genotypes. Amplification in one case employed a fluorescence-labeled primer; the other DNA's were amplified with standard primers. The double-strand, fluorescent probe for a wild-type homozygote, without further purification, was denatured and reannealed in the presence of a 30-fold excess of unlabeled PCR product from a wild-type homozygote (panel A) or a wild-type/variant heterozygote (panel B). The electropherograms depict the input sample (969 bp), cleavage fragment (95 bp), and a large excess of unincorporated, labeled primer to the left of the 95-bp peak. Per cent cleavage for each reaction is also provided in each panel.

FIG. 10 is a schematic representation illustrating genotyping of complex haplotypes in a self-annealing PCR-CRMS assay. PCR-CRMS was performed on 8 previously genotyped samples with complex SNP haplotypes within the promoter region and 5′UTR of CDKN1A. The haplotypes (A1, Ala, A2a, A3, C1, D1, and Dla; Geller et al., manuscript in preparation) are indicated on the left of the Figure. Relative location of the SNPs (homozygous variants: vertical bars; heterozygous variants: half-vertical bars) and the reporter sites (diamonds) are schematically represented in the middle portion of the Figure. At the fragment termini and over the SNP and reporter sites are numbers corresponding to a PAC reference clone DNA sequence (NCBI accession no. Z85996). At the right of the Figure are columns listing the percent digestion observed in the self-annealing assay, as well as the corresponding percent cleavage expected (theoretical maximum).

FIG. 11 is a graph showing MutL concentration correlation with the length of the target DNA. The optimal MutL enzyme concentration was determined for different target DNA's varying in size using a modified Taguchi optimization procedure (see Examples and Cobb et al., 1994). A linear correlation of the apparent concentration of MutL enzyme with the logarithm of PCR product length was observed. The correlation coefficient and derived equation are shown.

### DETAILED DESCRIPTION OF THE INVENTION

In conducting a study of genetic susceptibility to cancer in affected sibling pairs, the possibility of replacing microsatellite genotyping with a method based on mismatch detection was investigated. Accordingly, mismatch detection was modified to accommodate polymerase chain reaction (PCR) products bearing candidate genes and regions. The E. coli mismatch detection system, employing the factors MutS, MutL and MutH, was adapted for use in PCR-based, automated, high-throughput genotyping and mutation detection of genomic DNA.

Using an adaptation of the Taguchi method (Cobb et al., 1994), a comprehensive biochemical optimization of the above technique was conducted. The effects of such factors as choice of polymerase, monovalent cation concentration, ADP/ATP rations, and position of the MutH recognition signal have been quantified for different target DNA's ranging from 260 to 1250 bp. It was found that optimal experimental conditions for a given target region are independent of sequence environment and depend mostly on the PCR product size. Direct correlations of the KCl and Mut L concentrations with the length of the target DNA was observed. Therefore, near optimal assay conditions for a new target region were predictable based on the PCR product size.

The above-mentioned modifications, which are collectively designated as PCR-Candidate Region Mismatch Scanning (PCR-CRMS), have simplified the mismatch scanning assay, rendered it quantitative, and demonstrate its potential for cost-effective, high-throughput genotyping and mutation detection.

Although the invention is referred to as PCR-CRMS, methods of amplification other than PCR, such as strand displacement amplification (SDA) and ligase chain reaction (LCR), may be used for amplification of candidate regions.

Strategy for Adaptation of PCR-CRMS to High-Throughput Genotyping.

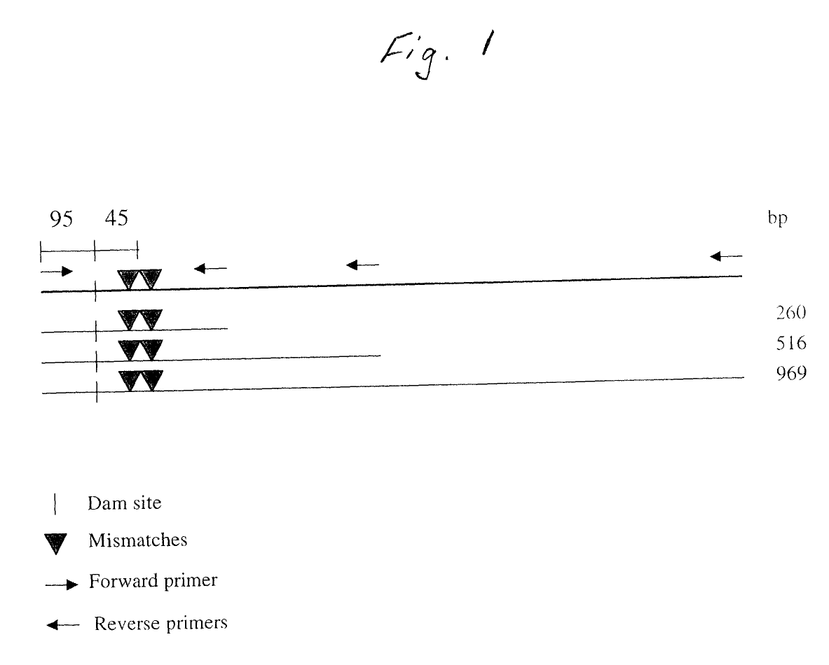

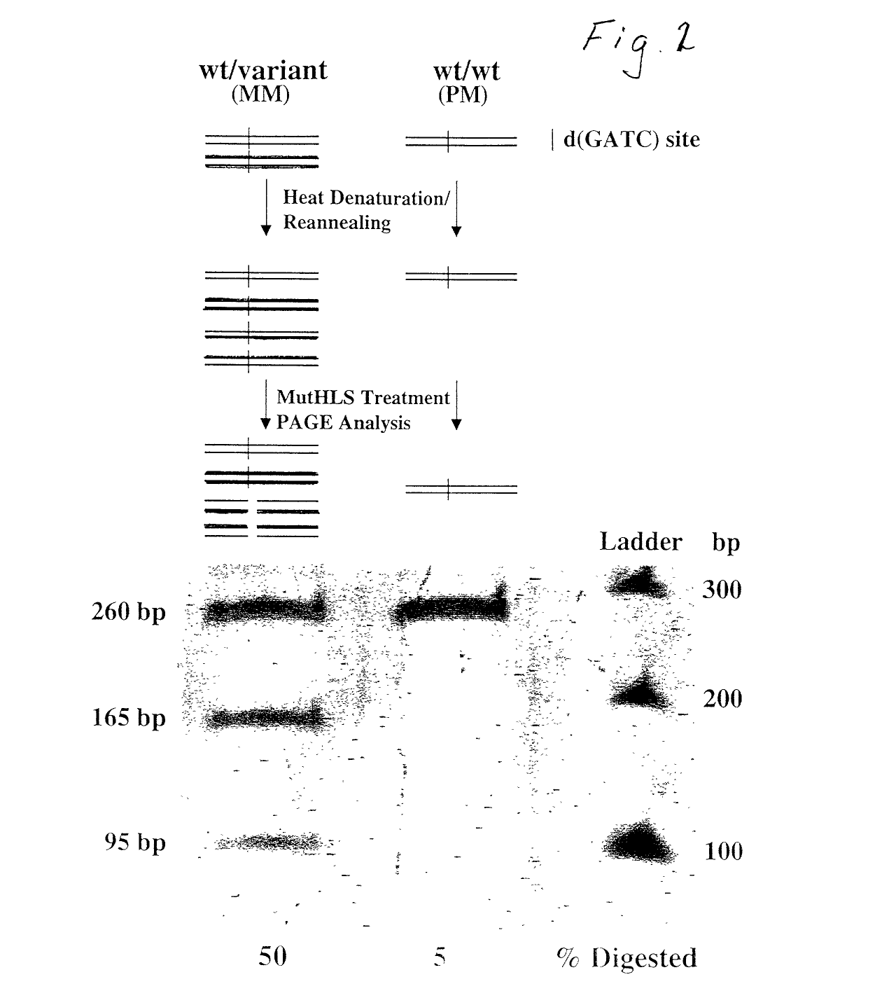

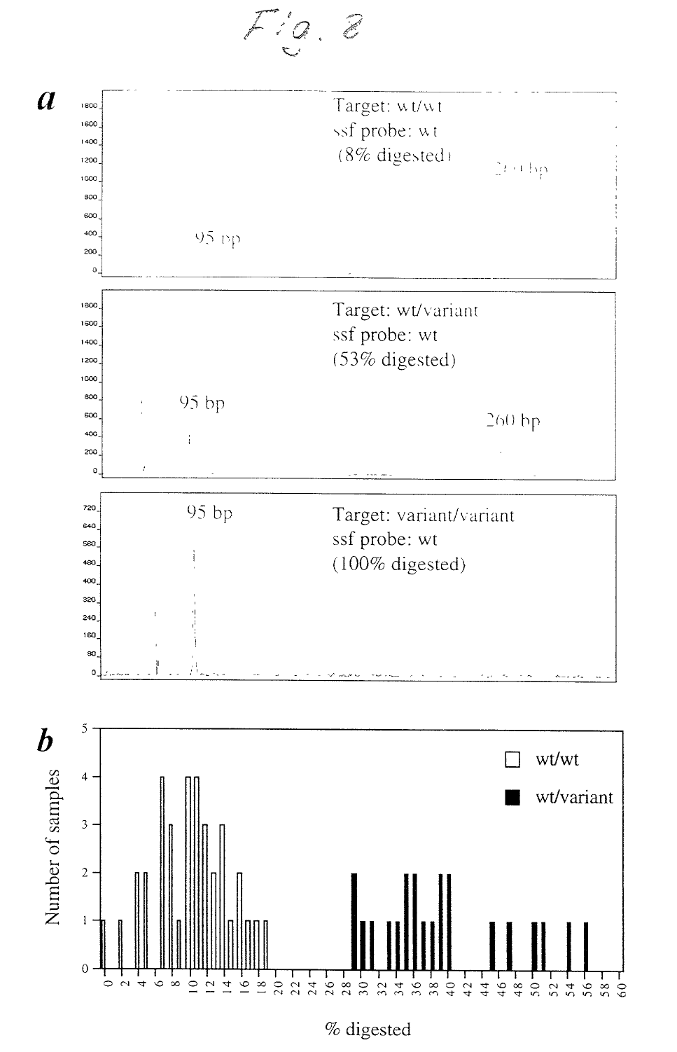

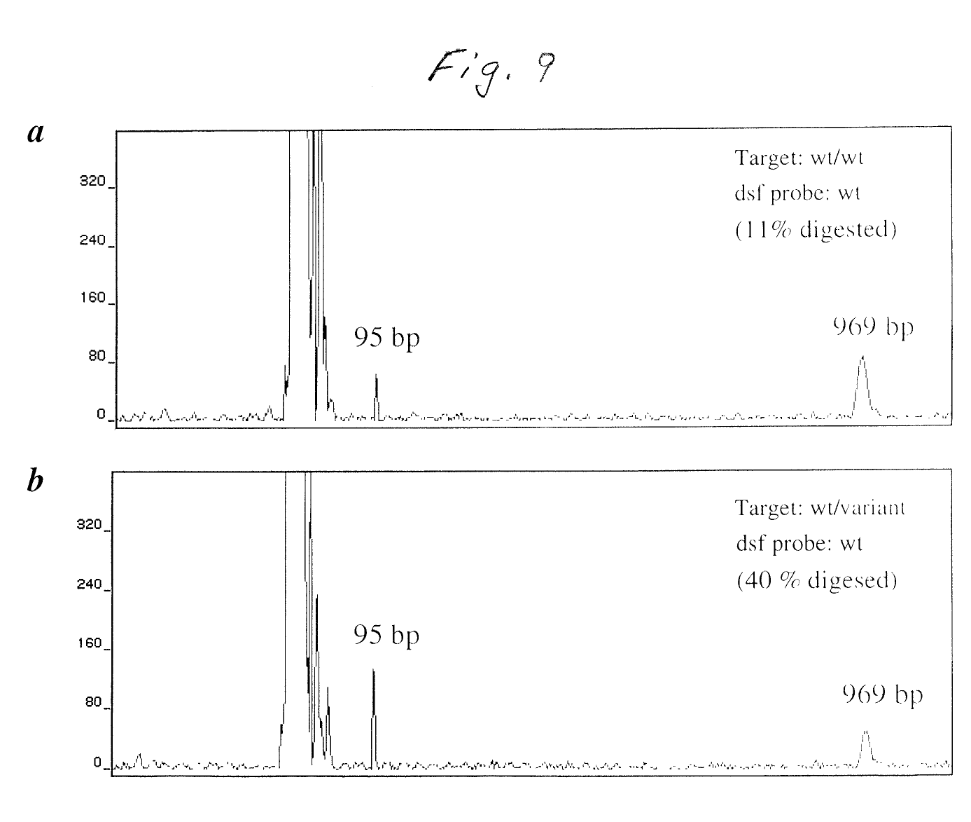

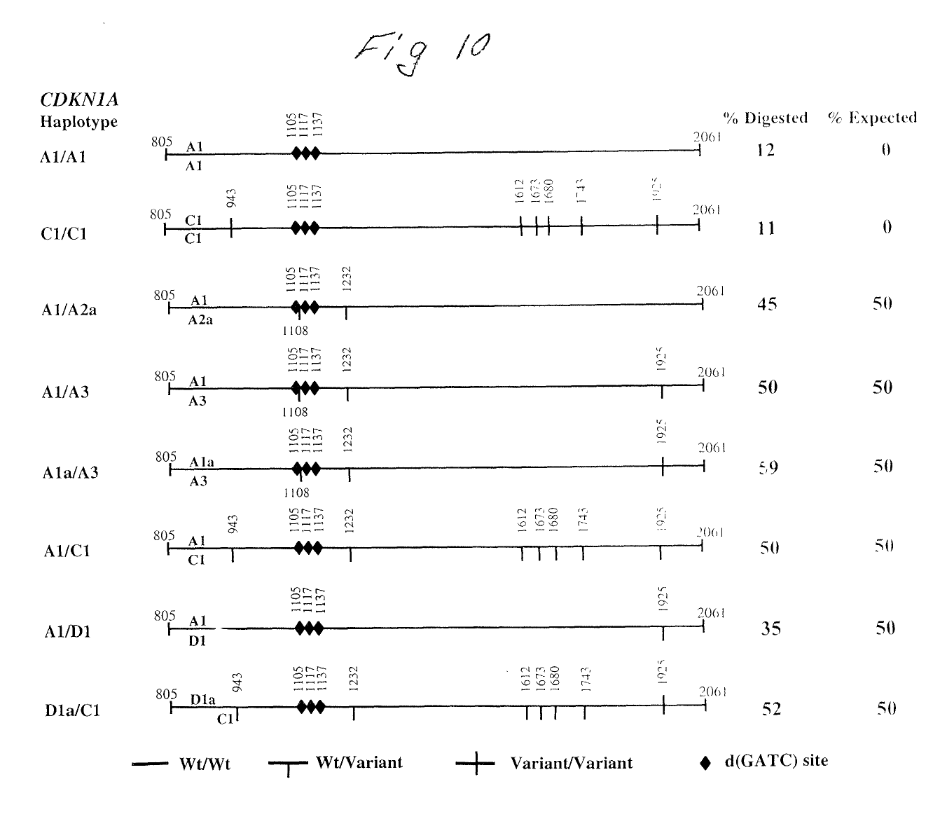

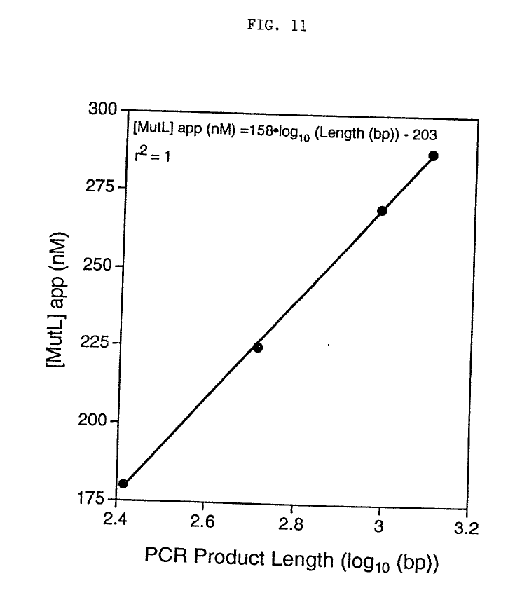

To test the feasibility of employing mismatch scanning for high-throughput genotyping, four stages of adaptation were examined. First, the terminal exon of CDKNIA was selected for the initial analysis (FIG. 1) because of the occurrence of suitable polymorphisms and dam sites, as well as the existence of a large number of DNA samples which had been previously genotyped by other methods. PCR-amplified DNA's from known homozygotes and heterozygotes were employed at the test locus; and the DNA's were denatured and reannealed individually before incubating them with mismatch enzymes. The fraction of expected hetero- and homoduplexes was predictable (FIG. 2); thus, this approach could be used to determine if quantitative results were possible. Second, the possibility was examined of using a fluorescence- labeled, single-strand, reference probe against target DNA's from patients and quantitating the results on an automated DNA sequencer (FIG. 8). Third, the potential for direct allele-sharing assays was investigated by directly testing the DNA of one relative against another (FIG. 9). Finally, the feasibility of using PCR-CRMS for analyzing regions bearing complex sets of haplotypes (FIG. 10) was examined.

PCR-CRMS using Self-Reannealed PCR Products (Mutation Detection Mode).

Following PCR amplification of test DNA's, aliquots of the products were denatured and reannealed individually. MutH, L, S were then added. The fraction of reannealed PCR product cleaved by MutH was obtained from a quantitative fluorescence scan of the polyacrylamide gel FIG. 2 shows a typical PCR-CRMS assay performed on 260-bp CDKNIA amplicons derived from previously genotyped homozygous (wt/wt) and heterozygous (wt/variant) individuals. Using a modified Taguchi protocol (Example 6), a goal was set for at least a 3:1 ratio of heterozygote to homozygote (background) cleavage and a heterozygote cleavage as close to the theoretical 50% as possible. In the example shown, only 5% of the homozygous sample was cleaved, while the heterozygous sample was cleaved at the 50% level expected. Thus, the signal-to-noise ratio was suitably high, while heterozygote recognition/cleavage were excellent. The conditions required for this performance are listed in Table 1. Optimal specificity required the preincubation of MutS and target DNA with ADP, as well as the addition of DMSO to the reaction.

TABLE 1
Optimized parameters for different target DNA sizes

| Target DNA (bp) | 260 | | 516 | | 969 | | 1257 5′UTR | |
|---|---|---|---|---|---|---|---|---|
| Analysis method | Mut. Det. | Genotyping | Mut. Det. | Genotyping | Mut. Det. | Genotyping | Mut. Det. | Genotyping |
| mM KCl | 85 | 60 | 95 | 75 | 110 | 95 | 122 | 102 |
| DMSO (%) | 5 | 5 | 0 | 0 | 0 | 0 | 0 | 0 |
| ADP (μM) | 100 | 10 | 0 | 0 | 0 | 0 | 0 | 0 |
| Mut L (nM) | 180 | 180 | 225 | 225 | 270 | 270 | 288 | 288 |

Fixed concentration of target DNA (25 nM), Mut H (85 nM), Mut S (275 nM) BSA (100 μg/ml), MgCl₂ (6 mM), DTT (1 mM),
Tris buffer (20 mM), EDTA (0.4 mM), ATP (1.5 mM), and Expand ™ buffer (0.3×) were used. Mut. Det: Mutation Detection Mode, Genotyping: Genotyping Mode.

When PCR-CRMS was first attempted with 516 and 969 bp PCR products at the same locus (FIG. 1), the experimental conditions optimal for the 260 bp target appeared inappropriate for the longer targets; it became necessary to re-optimize the assay for each target. The Taguchi method was again successfully applied to estimate the effects of individual components (Table 1 and FIG. 3). Interestingly, the larger targets did not require DMSO or preincubation with ADP. Moreover, it was apparent that the salt concentration was a key determinant in obtaining specificity. For example, the heterozygote/homozygote cleavage ratio obtained using the 969 bp target increased nearly 3-fold by simply increasing the KC1 concentration by 20 mM (FIG. 3B).

Factors Affecting Optimization of PCR-CRMS.

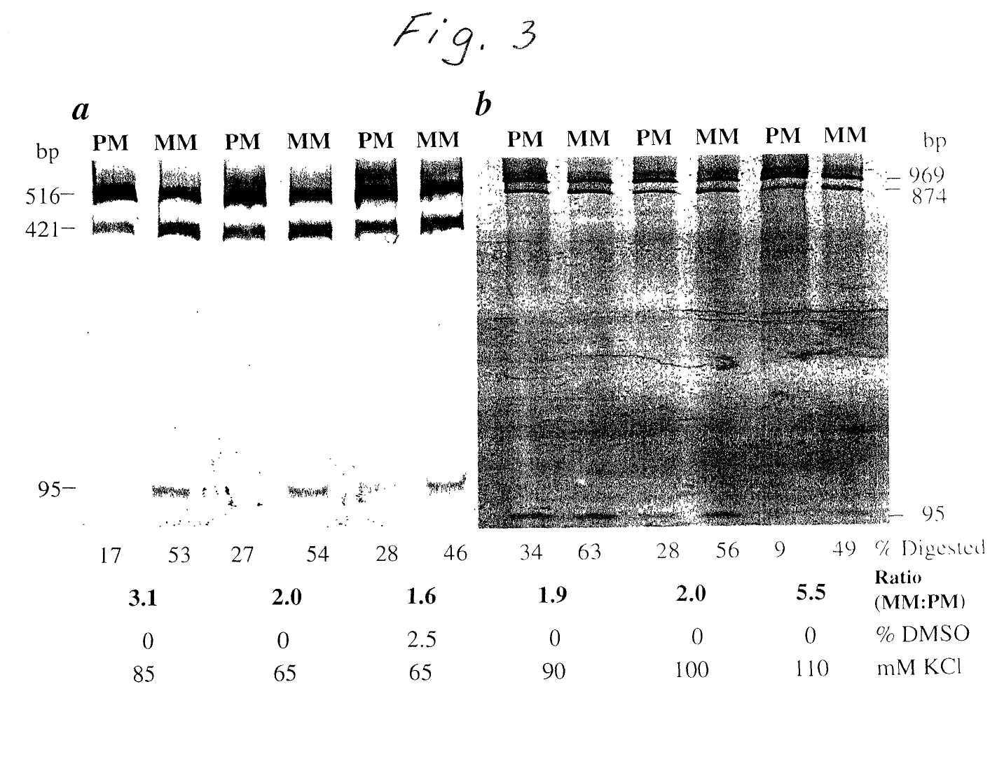

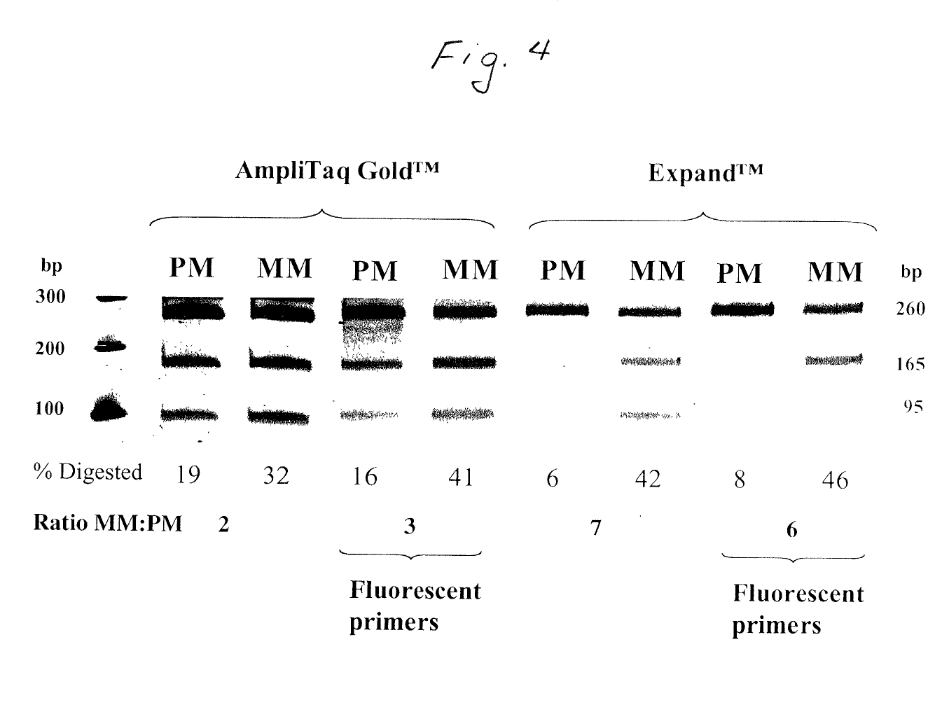

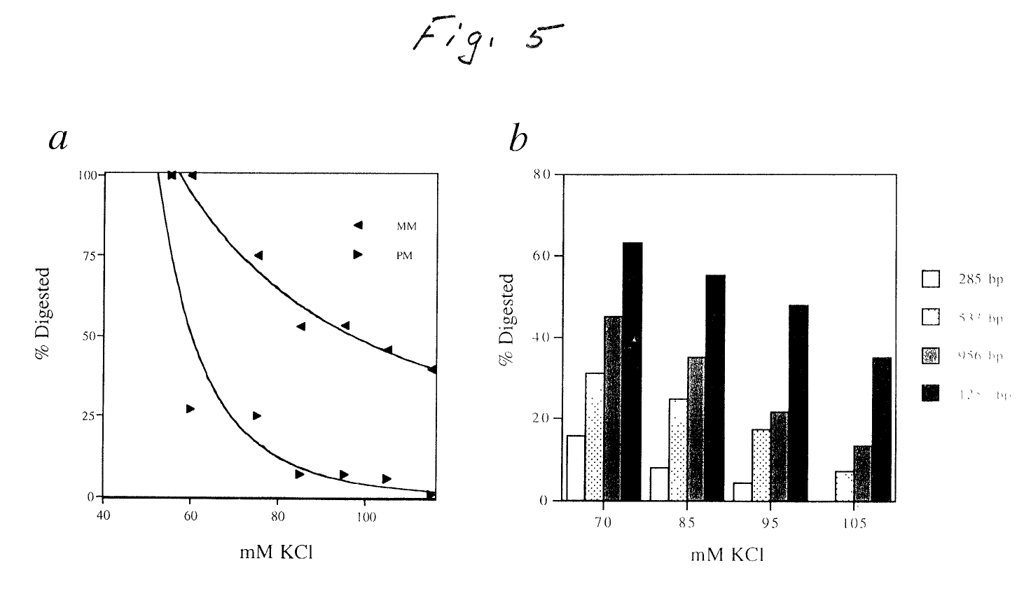

In addition to using Taguchi optimization of PCR-CRMS, the effects of several factors were investigated separately, which had appeared initially to be important for increasing the cleavage of true mismatches while maintaining low background cleavage rates. Because errors during PCR amplification could potentially increase the background mutH cleavage during mismatch scanning (Smith et al., 1996), a wide variety of thermostable DNA polymerases were first tested to establish which was best suited for target amplification and mutation detection. A comparison of the two best enzymes, AmpliTaqGold™ and the Expand™ High Fidelity enzyme cocktail, is shown in FIG. 4. Homozygous DNA amplified with AmpliTaqGold™ was cleaved at a 2- to 3-fold higher rate than that amplified with Expand™ High Fidelity. Therefore, it was confirmed that the error rate of polymerases contributed to a high background, with the Expand™ High Fidelity enzyme cocktail providing the degree of proofreading required for successful application of PCR-CRMS.

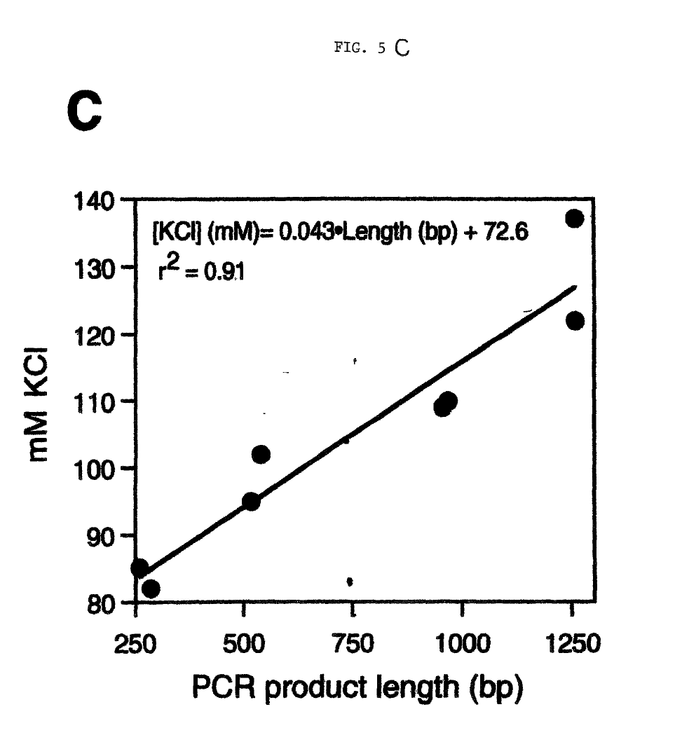

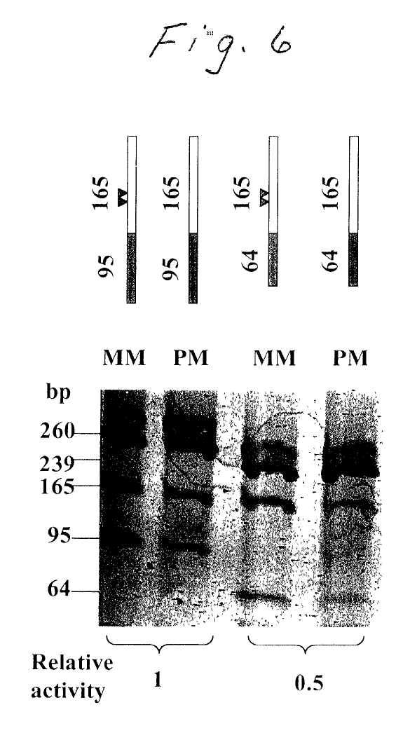

A series of experiments also were performed to titrate the optimal KCl concentration. The results of one such trial are depicted in FIG. 5A. At 55 mM total salt concentration, both the heteroduplex DNA target (mismatch; MM), as well as the homoduplex target (perfect match; PM) were entirely cleaved. The addition of only 5 mM KC1 (60 mM total) reduced the nonspecific cleavage of the PM DNA to 27%. At 85 mM both the MM cleavage (53%) and the specific:nonspecific ratio (53%:7%′ were optimal Another experiment, using four different target amplicons within a completely distinct locus (human androgen receptor), was performed using KCl concentration ranging from 70 mM to 105 mM (representing the linear range observed in FIG. 5A). The results (Figure SB) showed that a linear relationship existed between the measured background activity and the length of target fragment over a range from 285 to 1257 bp. Using the data pooled from Figure SB and Table 1, the KCl concentration was plotted, yielding optimal cleavage rate ratios against DNA fragment length. FIG. 5C shows a linear correlation (r²=0.91) from which Equation 1 was derived.

[KCl]_{10% max background level}0.043·Fragment length (bp)+72.6 mM. (Eq.1)

To assess the validity of the relation in Eq. 1, a PCR-CRMS assay was optimized for a new locus (38 bp long from human chromosome 22) using KCl concentration increments of 5 mM. The optimal salt concentration was experimentally determined to be at 90 mM (data not shown); that predicted from Eq. 1 was 89.2 mM.

Another parameter, the optimal amount of MutL, also demonstrated direct dependence on target length (Table 1 and FIG. 11). A direct correlation of the MutL optimum with the logarithm of the fragment size in base pairs (r²=1) was observed. Equation 2 was derived from these data.

[MutL]app.(nM)=158·log₁₀(Fragment length (bp))−203 (Eq.2)

Since the assays were performed using enzyme fractions with partial purity (one-step purification; see Examples), equation 2 was expressed as “apparent” concentration of mutL and must, therefore, be readjusted with new batches of enzyme. However, the knowledge of this correlation provides an important tool for easy optimization of new target regions.

Although optimization of PCR-CRMS with PCR products could be shown to lead to results quantitative enough for use in genotyping and allele sharing work, maximal flexibility of this method for genotyping requires one further condition: The efficiency of cleavage must remain high even if the dam (GATC) recognition site is very close to the end of the target DNA fragment. This flexibility in dam site location greatly increases the options for multiplex analysis and leaves open the possibility that the method may be further adapted to mass spectrometry. Others have shown that mutH cleavage efficiency drops dramatically as the dam site moves closer than 200 bp to the fragment end; under the reaction conditions employed in that study only 20% efficiency was possible at 200 bp (Smith et al., 1996). As shown in FIGS. 2 and 3, with optimization—particularly of the KC1 concentration—quantitative cleavage was obtained with the dam site 95 bp from the end. By moving the upstream primer closer to the dam site, the efficiency of cleavage was also tested when the site was at 64, 45, 30, and 15 bp away from the end. A 50% relative cleavage efficiency was still possible at 64 bp from the target fragment end (Table 2). At 45 bp relative cleavage dropped to 13%; no cleavage was detected with dam sites 30 and 15 bp from the end. Therefore, PCR-CRMS may still be useful in mutation detection applications with the dam site as little as about 45 bp from the end of the target fragment, but quantitative genotyping probably will require distances greater than about 64 bp.

TABLE 2
Effect of GATC site position on PCR-CRMS
efficiency

| | | | | | |
|---|---|---|---|---|---|
| Distance (bp) | 95 | 64 | 45 | 30 | 15 |
| Relative activity | 1 | 0.5 | 0.13 | BDL | BDL |

Distance (bp): Distance between the GATC site and the end of the PCR product.
BDL: Below Detection Limit

Fluorescence-Based PCR-CRMS (Genotyping Mode).

In preparing for a fluorescence-based assay, it was considered whether the incorporation of a primer with a fluorescent tag into a PCR-amplified target would increase the background cleavage because of possible nonspecific interaction of the fluor with mismatch enzymes. As shown in FIG. 4, labeled and unlabeled PCR products exhibited the same performance with PCR-CRMS. Therefore, the presence of a fluorescence label at one end of the PCR-amplified target DNA did not increase the nonspecific cleavage rate, making possible the use of PCR-CRMS on an automated detection platform such as the ABI 377™.

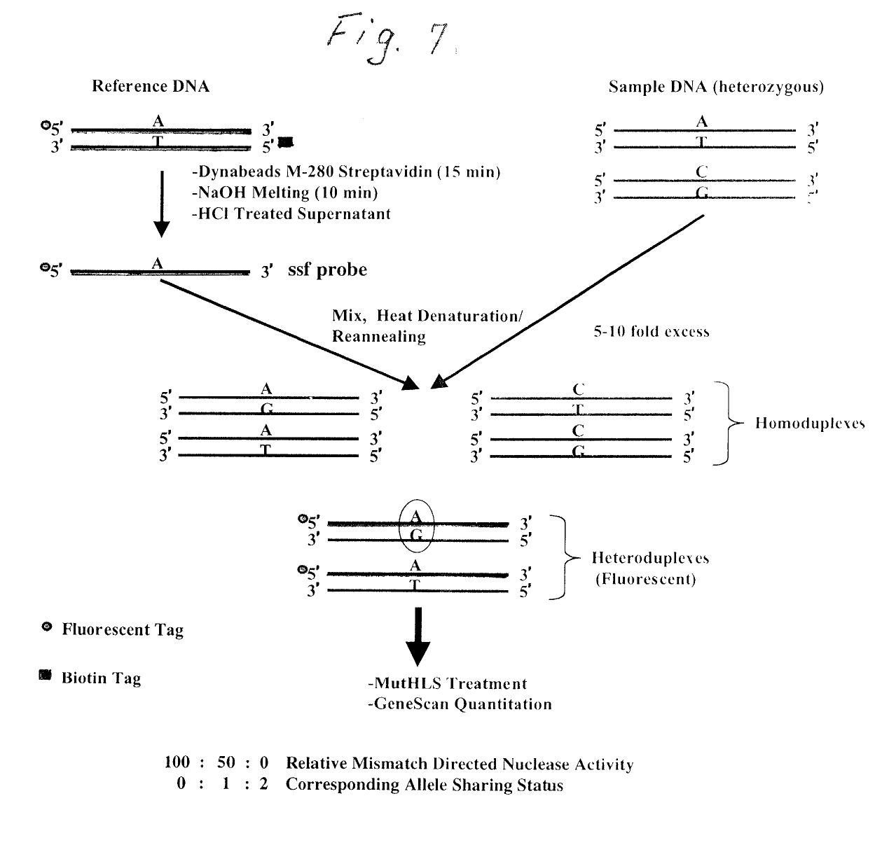

A schematic representation of a heteroduplex selection strategy using a labeled reference probe is shown in FIG. 7. The test DNA, from a locus of interest, is PCR-amplified from genomic DNA extracted from peripheral blood leukocytes. The reference probe is produced from DNA with a known genotype using the same primer sequences; however, one is tagged with a fluorescent label and the other with a biotin molecule. The single-stranded fluorescent probe (ssf probe) is purified using paramagnetic streptavidin beads and NaOH treatment. An aliquot of the probe is then mixed with the test sample PCR product in a ratio of 1:5-1:10. The solution is heat-denatured and reannealed in the assay buffer. Reannealing yields six different species: four unlabeled homoduplexes and two fluorescence-labeled heteroduplexes. Following MutH, MutL, and MutS treatment, the reaction products are loaded on an ABI377™ sequencing gel. Only labeled heteroduplexes are detected; quantitation of percent cleavage is obtained directly from the GeneScan™ electropherogram.

FIG. 8A depicts electropherograms from 3 target DNA's sampled with ssf probe prepared from the 260 bp amplicon of CDKN1A exon 3. One DNA was homozygous for the wild-type allele (top); one heterozygous for the wild-type and variant alleles (middle); and one homozygous for the variant allele (bottom). As predicted, only 8% of the probe was cleaved when hybridized with homozygous wild-type DNA (wt/wt) and 53% with heterozygous DNA (wt/v). The entire probe was cleaved in the presence of a homozygous variant DNA sample (v/v). This result indicated that the ssf probe assay could be quite quantitative, thereby leading to using a mismatch detection strategy for genotyping, as well as mutation detection.

To test the feasibility of using PCR-CRMS for the genotyping of a larger number of samples, fifty-eight human genomic DNA samples previously genotyped at CDKN1A exon 3 by DNA sequencing were assayed blindly. FIG. 8B depicts the frequency distribution of cleavage fractions obtained from each of these samples. All samples previously known to be homozygous for the wild type allele in the region interrogated were grouped together in the range of 1-19% cleavage (open bars). The heterozygous samples for that same region were also grouped together by the assay. However, in this instance all the heterozygous DNA samples were cleaved at a rate of 30-56% (solid bars). On average, in the setting of a multi-sample assay, homozygous DNA's were cleaved at a rate of 10% compared to 40% for the heterozygous DNA's.

The transition to a fluorescence-based assay required further optimization of the reaction conditions. While longer targets could be assayed by the heteroduplex selection strategy, using the corresponding ssf probe and the ABI 377™ automated sequencer, the KCl concentration for a given target fragment size was always reduced by 10 to 20 mM when compared to self-annealing assays with unlabeled probes. Table 1 summarizes this and other parameters requiring further optimization for the automated assay.

Direct Fluorescence-Based Genotyping with Patient (sib-sib) Samples.

Throughput and flexibility of genotyping might be increased by labeling one sample DNA and testing it against another sample DNA (e.g., a family member), without the use of a reference probe. In this fashion DNA's from family members, such as affected siblings, could be compared to one another for the accumulation of allele-sharing data. To evaluate the performance of PCR-CRMS under these conditions, an assay was performed in which an unpurified, double-stranded, fluorescent probe (dsf probe) was prepared by amplifying genomic DNA from a previously-known homozygote. This DNA was reannealed with crude PCR products from genomic DNA of a homozygote (FIG. 9A) or heterozygote (FIG. 9B) in the ratio of 1:30, and PCR-CRMS was performed. As shown in FIG. 9, a simple dilution of unpurified, dsf probe was sufficient for maintaining the quantitative aspect of the assay. The cleavage of labeled probe in the presence of a homozygous sample was only 11%; the heterozygous sample cleavage was 40%, for a MM:PM ratio of nearly 4. This result demonstrated the feasibility of comparing relatives using PCR-CRMS.

Determination of Allele-Sharing Status in Complex Haplotypes.

To assess the reliability of the assay in quantitating differences among complex haplotypes, the target sequence was moved upstream to the promoter region of CDKNIA. A 1257 bp fragment at that location contained three GATC sites, as well as eight SNPs. These SNPs defined seven haplotypes (Geller et al., in preparation). Either self-annealing of homo- and heterozygote DNA samples (FIG. 10) or a heteroduplex selection assay that employed haplotype A1 as the ssf probe (not shown) was used; both methods gave equivalent results.

Eight different genomic DNA samples previously haplotyped at the CDKN1A promoter region were PCR-amplified. As expected, the two homozygous DNA samples, A1/A1 and C1/C1, were cleaved at the lowest rates of 11 and 12%. Five of six heterozygous samples (A1/A2a, A1/A3, Ala/A3, A1/C1, Dla/C1) were cleaved at an average rate of 51% (FIG. 10). Only one heterozygous genotype, carrying a SNP almost 900 bp away from the nearest reporter site, was cleaved at a lower rate of 35%. This rate was probably attributed to the relatively long distance separating the mismatch from the nearest available GATC site. PCR-CRMS thus is effective even when multiple SNPs and reporter sites are present in the target sequence under analysis.

The E. coli mismatch detection enzymes, MutS, MutL, and MutH thus also may be employed for quantitative genotyping of patient DNA samples. The method is easily adapted to automated sequencers for high-throughput usage; massed-tagged primers can, in principle, enable adaptation to genotyping by mass spectroscopy, as well. Thus, PCR-CRMS may supplement or even replace microsatellite genotyping in family-based genetic analyses such as genome scans of affected sibling pairs. The successful adaptation of mismatch scanning described herein coincides with the appearance of comprehensive human genome sequence, allowing the choice of any marker region coupled to a reporter dam site at any desired position in the PCR fragment. Although the commercial availability of the required mismatch enzymes has been unreliable in the past, all three proteins were purified successfully to near homogeneity by using a simpler one-step, Ni²+-chelation affinity batch protocol. Therefore, PCR-CRMS is accessible to individual research laboratories, as well as to academic or commercial consortia.

Several roadblocks to the efficient use of mismatch scanning for genotyping have been examined: (1) the effect of polymerase errors during PCR amplification of genomic target regions on background levels of mismatch detection; (2) the degree of difficulty in optimizing the mismatch detection reaction for each new genome segment scanned; (3) the placement of dam recognition sites in regions convenient for analysis without loss of MutH cleavage efficiency; and (4) the isolation of intersample heteroduplexes for analysis without employing complex selection strategies. Each of these difficulties may be overcome with straightforward solutions that are amenable to automation.

For example, previous reports on the use of MutS (Lishanski et al., 1994) or MutHLS (Smith et al., 1996) enzymes for mutation detection of PCR products revealed conflicting results on the use of different DNA polymerases for target amplification. One report suggested that Pfu polymerase does not perform better than Taq polymerase in terms of the signal-to-noise ratio. This was unexpected since Taq DNA polymerase lacks 3′→5′ proofreading exonuclease activity and, therefore, exhibits a higher misincorporation rate (Eckert et al., 1991; Tindall et al., 1988). A second report examined the effect of using Pfu Vent or Taq polymerases and found almost a direct correlation of MutHLS nonspecific cleavage of homozygous DNA samples with the previously established frequency of polymerase errors during PCR amplification. To determine the best conditions for PCR-CRMS, the cleavage activity was measured on PCR products amplified with the AmpliTaqGold™ and the Expand™ High Fidelity enzyme systems. The latter system is composed of an enzyme mix containing thermostable Taq and Pwo DNA polymerases and is designed to give PCR products from genomic DNA with high yield and fidelity (Barnes et al., 1994). Results obtained using PCR-CRMS confirmed that the Expand™ system, with an error rate of 8.5×10⁻⁶, was superior to AmpliTaqGold™, with an error rate of 2.6×10⁻⁵. This result set a minimally acceptable level of replication error for satisfactory application of PCR-CRMS.

An important conclusion from these studies is that optimization of the mismatch detection reaction is dependent upon salt and mutL concentration in a predictable relationship to target fragment length. A previous report, using human repair enzymes, demonstrated a clear maximum of repair at 130 mM KCl, with the efficiency of the reaction dropping precipitously at both lower and higher salt concentrations (Blackwell et al., 1998). Using experimental conditions with bacterial enzymes and the 260 bp target, it appears that a higher salt concentration (150 mM) results in a low efficiency of cleavage. The use of a smaller amount of KCl (60 mM) was too permissive, and the specificity for mismatch was lost. Both homozygous and heterozygous samples were cleaved without discrimination. The optimal salt concentration for the 260 bp fragment was 85 MM. This result was in agreement with a previous study postulating weaker hMutSα·DNA interaction with increasing salt concentration (Blackwell et al., 1998). The same group demonstrated that at low KCl concentration (<50 mM), both homo- and heteroduplex DNA activated hMutSα ATPase activity to the same degree. By analogy to the human enzyme, it seems that the regulation of the bacterial MutH cleavage discrimination by KCl was effective at the MutS·DNA binding step.

The optimal experimental conditions for longer PCR products amplified from the CDKNIA locus differed mainly by a proportionally higher salt concentration. This was unexpected, since the mismatches and reporter site sequence environment used were identical. In addition, when different regions of the human androgen receptor gene locus were tested, the same correlation was observed. These results were in agreement with data reported by Blackwell et al., 1998, where they found a significant and reproducible DNA chain length effect on k_{cat,ATP} of hMutSα. The authors suggested that a proportional increase in the ATPase activity with the DNA chain length was the expected result for the translocation mechanism model (Blackwell et al., 1998; Young et al., 1994; Young et al., 1994; Blackwell et al., 1998), rather than the molecular switch model (Gradia et al., 1997). Under experimental conditions, the interrogation of longer PCR products resulted in higher background cleavage activity, which seemed consistent with the hypothesis that MutS locates the target mismatch by moving along the DNA helix following a translocation mechanism. On the other hand, this result was inconsistent with the switch model, which does not invoke a functional role for sequences external to the mismatch.

The results obtained following optimization using the Taguchi method, done on targets differing by their size and sequence environment, demonstrated the necessity of determining the range of MutL concentration to be used for optimal cleavage of mismatch-containing DNA, while keeping the background activity as low as possible. A direct logarithmic correlation (r²=1) was found between the length of the target and the optimal MutL concentration. As observed with the salt concentration effect, this phenomenon was independent of the sequence environment. This direct relation with the substrate length further supported the idea that a translocation mechanism was involved in mismatch scanning.

Two previous studies (Ban et al., 1998; Hall et al., 1999) revealed that MutL alone could stimulate some MutH endonuclease activity via direct physical interaction in the presence of ATP. These studies also demonstrated that the addition of MutS to the reaction further stimulated MutH activity, specifically for mismatch-containing DNA. Therefore, the basal activity measured on perfectly-matched DNA samples was explained by the spontaneous capacity of MutL to activate MutH without MutS, emphasizing the importance of determining the appropriate concentration of MutL to obtain quantitative genotype and allel-sharing information. The Taguchi method was particularly suited to these determinations.

The relative placement of the GATC recognition sequence may be an important determinant of PCR-CRMS flexibility. Keeping the cleavage close to the end of the target fragment is important if the technique is to be adapted to mass spectrometry, where current technology is optimal for fragments smaller than 100-120 nucleotides in length. Detection of target fragment cleavage that signals heteroduplex mismatch is possible for a wide range of fragment sizes on the automated sequencer when appropriate changes in gel matrix are employed. The present studies demonstrate that cleavage efficiency can be maintained when the dam site is within about 85-100 nucleotides of the end of the target fragment. Much shorter distances may be employed if mutation detection, rather than genotyping, is the goal.

The adaptability of mismatch scanning for genotyping may depend on whether intersample heteroduplex selection can be accomplished in a manner simple enough for automation. As shown herein, two different schemes that involve diluting out a labeled reference strand with PCR products from other test DNA's may be adopted. One reference probe may be used for many samples, or one sample of each relative pair may serve as the reference. Either approach, even in combination, appears promising for high-throughput genotyping.

As discussed earlier, one type of mismatch that cannot be effectively detected is C-C mispairing. Two recent publications have demonstrated that the point mutation, C→G, is one of the least frequent occurring in the human genome at 4.7-5.0% of events (Krawczak et al. 1998; Hawkins et al., 1997). For mutation detection purposes, this means that, on average, only 1 of 20 mutations present in the human genome would not be detected by using a single probe. However, the possibility of interrogating the other strand, where a C→G mutation would be seen as a G-G mismatch, should alleviate this problem. The use of a probe labeled on both ends with different dyes should be feasible, so that both strands can be interrogated at once. This strategy should further improve the sensitivity of the method by detecting virtually all type of mutations. In any event, for genotyping purposes, lack of C-C mismatch detection is not an issue, since other variants would suffice.

Finally, a major attraction of mismatch scanning in the characterization of human genetic variation is the ability to exploit such variation with minimal effort, especially when contrasted with microsatellite genotyping. This ease of use, together with the expectation of a very high density of useful SNPs in human genomic sequence (Halushka et al., 1999; Cargill et al., 1999), will provide great flexibility in the choice of target regions for PCR-CRMS genotyping.

Although fluorophore-labeled probes were used herein, it is readily recognized that probes can be labeled with any type of chemicals that will not interfere with the mutation detection enzyme, e.g., a radioactive component, a mass tag, an chemiluminescent component, a magnetic component, etc.

A double labeled probe (PCR product labeled on both ends) can be used to interrogate both strands at once and to detect all type of mutations. A strategy using a double labeled probe (dl probe) would be basically the same as using a double strand probe labeled at one end (on one strand). The difference is that the dl probe could be obtained following amplification by PCR of the candidate region using differently labeled forward and reverse primers. The use of dl probe will increase the sensitivity of the method by virtually detecting all type of mutations. For example, the C-C mismatches are poorly detected by the system. However, by labeling one strand of the probe with one dye and labeling the other strand with a different dye, the C-C mismatch will be seen as a G-G mismatch, which is a good substrate for the mismatch enzymes. In addition, the use of a dl probe will increase the accuracy of genotyping by duplicating the analysis.

Multiplexing can be achieved by incorporating multiple PCR products in the same test tube, microwell or other container. Individual mutation detection and genotype determination can be done by selecting different sizes of the target DNA's and cleavage products. Secondly, differentiation can be further achieved by amplifying different loci with different tags, e.g., fluorescent dyes, mass tags, radioactivity.

To perform automation using robotics, mineral oil may be used to prevent evaporation. Since there are multiple reactions steps involving incubation in moderate to high temperature, and the use of 96 or 384 well plates, currently available in the market, could not be efficiently sealed and/or while the robot arm is adding reagents to the other wells, inert mineral oil will be added on top of the solutions to prevent evaporation.

In accordance with the invention, the candidate region analyzed may contain more than one polymorphism. Additionally, multiple candidate regions may be mismatch scanned either separately or simultaneously in the same container, including by amplifying candidate regions of different sizes that produce cleavage products of different sizes, or by amplifying different candidate regions with different labels.

In a preferred embodiment, the amplification step may be carried out with a radioactive- or fluorescent-labeled primer or alternatively, mass-tagged primers.

After amplification of the DNA sample, the amplified DNA may be denatured and reannealed in an assay buffer under suitable conditions and for a suitable amount of time, at about 90° C. to about 110° C. and preferably at about 99° C. for about 10 minutes and at about 50° C. to about 70° C. and preferably at about 60° C. for about 15 minutes, the buffer containing (i) about 60 mM to about 100 mM, preferably about 70 mM to about 90 mM, more preferably about 80 mM to about 90 mM, and most preferably about 85 mM potassium chloride when the candidate region is about 260 or fewer bp, and proportionately higher concentration ranges of potassium chloride when the candidate region is more than about 260 bp (see Table 1); and (ii) 80 μM to about 120 μM, and preferably about 90 μM to about 110 μM, and most preferably about 100 μM ADP and about 4% to about 6% and preferably about 5% DMSO when the candidate region is about 260 or fewer bp, less or no ADP and DMSO when the candidate region is more than about 260 bp, and no, or substantially no, ADP or DMSO when the candidate region is about 516 or more bp.

Digestion of the reannealed DNA may then be carried out in the assay buffer containing a suitable amount of DNA (preferably about 20 nM to about 30 nM, and most preferably about 25 nM DNA), a suitable amount of MutH (preferably about 80 nM to about 90 nM, and most preferably about 85 nM MutH), a suitable amount of MutS (preferably about 250 nM to about 300 nM, more preferably about 260 nM to about 290 nM, and most preferably about 275 nM MutS), a suitable amount of ATP (preferably about 1.0 mM to about 2.0 mM, more preferably 1.3 mM to about 1.7 mM, and most preferably about 1.5 mM ATP), and a suitable amount of MutL (preferably about 160 nM to about 200 nM, more preferably about 170 nM to about 190 nM, and most preferably about 180 nM MutL) when the candidate region is about 260 or fewer base pairs, and proportionately higher concentration ranges of MutL when the candidate region is more than about 260 bp (see Table 1).

In a preferred embodiment, the 5′ GATC 3′ site may be at least about 45 bp, more preferably at least about 64 bp, and most preferably at least about 95 bp, from the end of the candidate region or target fragment (see Table 2). A preferred distance is within about 85 to about 100 bp from the end of the target fragment The mismatch and 51 GATC 3′ sites may be separated by any suitable distance along the DNA, preferably by up to about 1 kB.

Suitable conditions, amounts, amounts of time and/or distances may be determined either empirically with the guidance provided herein or based on the knowledge in the art, including the disclosures incorporated herein by reference.

The fraction of DNA cleaved may be determined by electrophoresing the digested DNA and quantitating the resulting bands, or alternatively, by mass spectroscopy.

In other preferred embodiments, the DNA polymerase used for PCR may be a mixture of the thermostable Taq and Pwo polymerases.

In other preferred embodiments in which a probe is utilized, the probe may a single-stranded probe that is labeled at one end, a double-stranded probe that is labeled at one end of one strand, or a double-stranded probe that is labeled at one end of one strand with one label and at one end of the opposite strand with a different label.

In other preferred embodiments, the fraction digested of the single stranded labeled probe may be determined by electrophoresing the digested DNA on an automated DNA sequencer and the fraction digested may be quantitated from electropherograms.

In other preferred embodiments, one or more of the reaction steps may be carried out in multiple well plates, and more preferably under inert mineral oil to prevent evaporation.

In still other preferred embodiments, the labeled amplified DNA may be labeled during amplification by use of a labeled primer or a pair of labeled primers. Additionally, the labeled amplified DNA may be labeled during amplification by use of a 5′-fluorescent labeled primer or a pair of 5′-fluorescent labeled primers. The labeled DNA also may be mixed with unlabeled DNA in a ratio in the range of about 1:5 to about 1:30.

The invention is further illustrated by the following examples, which are not intended to be limiting.

Materials.

His-Bind Quick Columns were purchased from Novagen Inc., Madison, Wis. The Centriplus concentrators were from Amicon, Inc., Beverly, Mass. The QIAquick PCR Purification Kit was from Qiagen Inc. (Valencia, Calif.). Dynabeads M-280 Streptavidin was purchased from Dynal A.S. (Oslo, Norway). The AmpliTaq Gold™ was purchased from Roche Pharmaceuticals. The Expand™ High Fidelity PCR System, ATP (lithium salt), ADP and IPTG were obtained from Boehringer Mannheim. Genescan-500 (TAMRA) size standards were purchased from PE Applied Biosystems. The CDKNIA PAC clone 431A14 was obtained from the Roswell Park Cancer Institute (Buffalo, N.Y.).

### EXAMPLE 1

Expression of the His₆-MutHLS Proteins. A modification of the procedure provided by Feng and Winkler was used (Feng et al., 1995). Briefly, a fresh colony of each strain (TX3149, TX3150 or TX3151) was used to inoculate 40 ml LB medium containing 50 μg/ml carbenicillin. The culture was incubated at 37° C. and shaken at 250 rpm for 6-8 hr to mid-exponential phase (OD₆₆₀ₙₘ of 0.5-0.6). The cells were then harvested by centrifugation at 5000× g for 10 min, and the pellets were resuspended in 4 ml of LB medium for overnight storage at 4° C. The next morning 2×2.5 L flasks, containing 500 ml LB medium with 10μg/ml carbenicillin, were inoculated using 2 ml of the cell preparation from the previous evening. The cell suspension was incubated at 37° C. with rotary shaking (250 rpm) until the OD₆₆₀ₙₘ reached 0.5-0.6 (4-6 hr). Then 120 mg IPTG (1 mM final) was added and the cells were allowed to overexpress the recombinant proteins for 3 hr. The culture was then chilled on ice for 5 min and then centrifugated at 5000× g for 20 min at 4° C. The pellets were pooled and washed twice with 50 ml of ice-cold water following a final centrifugation at 3000× g for 10 min at 4° C. The pellet was then stored at −70° C. until the protein purification step.

### EXAMPLE 2

Large-Scale One Step Purification of the His₆-MutHLS Proteins.

All steps were carried out at 4° C. or on ice. The frozen cell extract was thawed on ice using 60 ml of 1× binding buffer (5 mM imidazole, 500 mM NaCl, 20 mM Tris-HCl pH 7.9)supplemented with protease inhibitors PMSF (1 mM), Antipain (50 μg/ml), Benzamidine (1 mM), leupeptin (2.5 μg/ml), and pepstatin A (2.5 μg/ml). Cells were disrupted using an ultrasound sonicator (4×20 sec) at 40% power level and 50% pulse (Sonifier 450, BRANSON). The cell lysate was then centrifugated at 35,000 × g for 20 min at 4° C. The cell-extract supernates were filtered using a 60 ml syringe and a 0.45 μ filter. The filtrates for MutS and MutL (60 ml) were then loaded onto two pre-equilibrated His-Bind Quick Columns (30 ml filtrate each column) (Novagen Inc., Madison, Wis.) following the manufacturer's instructions. For MutH, four columns were loaded using 15 ml filtrate per column. The columns were then equilibrated using 30 ml of 1× binding buffer and washed once with 50 ml of a solution wash buffer (60 mM imidazole, 500 mM NaCl, 20 mM Tris-HCl pH 7.9): binding buffer (1:1) and further washed using 13 ml of a solution of wash buffer: binding buffer (3:1). The his-tagged proteins were then eluted twice with 7 ml of a solution of elution buffer (300 mM imidazole, 500 mM NaCl, 20 mM Tris-HCl pH 7.9). The eluted fractions were concentrated for 30-60 min using Centriplus YM-50 concentrators (Amicon Inc., Beverly, Mass.). The buffer was then changed, using a NAP-25 column (Pharmatia) with buffer A: 20 mM Tris-HCl pH 8, 1 mM EDTA, 1 mM DTT, 200 mM KCl and 20% glycerol. 2.5 ml of Buffer B (Buffer B containing 94% glycerol instead of 20%) was added to the eluted protein solutions (3.75 ml). The final buffer composition of the protein samples were 50% glycerol, 20 mM Tris-HCl pH 8, 1 mM EDTA, 1 mM DTT, and 200 mM KCl. The enzyme preparations were then aliquoted and stored at −70° C.

### EXAMPLE 3

Amplification of Target DNA.

Target DNA's as well as the reference DNA were PCR-amplified, the latter using a FAM-labeled forward primer and a biotin-labeled reverse primer. The locus chosen as target to optimize the method included part of intron 2, exon 3, and the proximal 3′UTR of the human CDKNIA gene (FIG. 1). This region was selected for the presence of known RFLPs (Law et al., 1995; Larson et al., manuscript in preparation) and the availability of several, previously genotyped, human genomic DNA samples. The forward primer sequence used was 5′-TCTCAGTTGGGCAGCTCCG-3′. The reverse primer sequence for the 260 bp target was 5′-GCCAGGGTATGTACATGAGGAG-3′; for the 516 bp target, 5¹-CGCCTGTGACAGCGATGG-3′; and for the 969 bp target, 5′-GCTGAGAGGGTACTGAAGGGA-3′. For amplification of the target with a GATC site 64 bp from the 5′ end of the fragment, the forward primer sequence was, 5′-TCTTCTTGGCCTGGCTGAC-3′. The 260 bp PCR amplification was performed in a total volume of 20 μl, using 200 μM dNTPs, 250 nM of each primer, 1.5 mM MgCl₂, and 25 ng of DNA. Either AmpliTaqGold™ (PE Applied Biosystems) or the Expand™ High Fidelity enzyme preparation (Roche Pharmaceuticals) was used in the buffers provided by the vendor. PCR reactions were carried out with a first cycle of 96° C.-2 min, 60° C.-45 sec. 72° C.-45 sec, and 26-29 more cycles of: 94° C.-30 sec, 60° C.-45 sec, and 72° C.-45 sec, and a 3 min final extension. For the 516 bp and 969 bp amplicons, a final MgCl₂ concentration of 2.1 mM was used along with the Expand™ High Fidelity PCR System (Boehringer Mannheim). The 516 bp product was amplified with a first cycle of 96° C. for 2 min and 68° C. for 1 min, and 26-29 more cycles at 94° C.-30 sec, and 68° C.-1 min. The 969 bp amplicon was obtained using the previous PCR conditions with an annealing-extension time of 1 min 20 sec.

The promoter-5′UTR region of CDKN1A was amplified using the forward primer sequence, 5′CTGCTCCACCGCACTCTGG3′, and the reverse primer, 5′TCCGCTCCCATCTACCTCAC3′. Amplification was performed using the Expand High™ Fidelity enzyme preparation along with the buffer supplied by the manufacturer. The cycling conditions were: one cycle at 96° C. for 2 min and 68° C. for 1 min 40 sec, followed by 29 more cycles at 94° C.-30 sec, 68° C.-1 min 40 sec, and a 3 min final extension at 68° C.

Three different regions of the androgen receptor locus, varying in length and nucleotide sequence, were amplified as PCR-CRMS targets from a single male DNA sample: exon 5 (285 bp), the 5′UTR (537 bp) and exon 1 (956 bp). The forward primers were, respectively,

5′-CAACCCGTCAGTACCCAGACTGACC-3′,
5′-AAGGCAGTCAGGTCTTCAGTAGC-3, and
5′-CACTTGCATCTGCCACCTTTAC-3′;
and the reverse primers were
5′-AGCTTCACTGTCACCCCATCACCATC-3′,
5′-CACTTCGCGCACGCTCTG-3′, and
5′-GGAGGTGGAGAGCAAATGCA-3′.

Amplification was performed using the Expand™ High Fidelity enzyme preparation along with the buffer supplied by the manufacturer, supplemented with 500 ng/μl BSA. The cycling conditions were as follows: an initial cycle of 96° C. for 2 min followed by 45 seconds at the annealing temperature of 62° C. (285 and 537 bp targets) or 60° C. (956 bp targets), and 72° C. for 40 sec (285 bp), 45 sec (537 bp), or 1 min, 30 sec (956 bp). Thirty one cycles followed at 94° C., 30 sec; at the annealing temperature above for 45 sec (285 and 537 bp) or 1 min (956 bp), and at 72° C. for 40 sec (285 bp), 45 sec (537 bp), or 1 min, 30 sec (956 bp). All the reactions were ended with a 3 min final extension at 72° C.

PCR Primers for Investigating the Effects of GATC Position on PCR-CRMS Efficiency.

The reverse primer used was the same as for the 260 bp target DNA amplification. For amplification of targets with a GATC site 64, 45, 30 and 15 bp from the end of the fragment, the forward primer sequence were,

5′-TCTTCTTGGCCTGGCTGAC-3′,
5′-TTCTGCTGTCTCTCCTCAGATTTC-3′,
5′-TCAGATTTCTACCACTCCAAACG-3′, and
5′-TCCAAACGCCGGCTGACT-3′, respectively.

Single-Strand, Fluorescent DNA Probe (ssf Probe) Preparation.

For the purpose of heteroduplex selection, and detection of homozygote variations, we purified a single strand fluorescent DNA probe (ssf probe). This was achieved using high affinity binding of the unwanted biotin containing DNA strand to streptavidin beads. 40 μ1 of the PCR product harboring a FAM molecule at the 5 ′end of one strand and a biotin molecule at the 5′ end of the complementary strand, was first purified from salts and residual primers using the QIAquick PCR purification kit (Qiagen) using the manufacturer's protocol. DNA was eluted in 35 μ1 of elution buffer (EB) (10 mM Tris-HC1 pH 8.5). The ssf probe was purified using Dynabeads M-280 Streptavidin (Dynal). Briefly, the paramagnetic beads (17.5 μ1) were equilibrated in 17.5 μ1 of the 2× binding buffer (BB) (10 mM Tris-HC1 pH 7.8, 1 mM EDTA, 2 M NaC1). The beads, resuspended in 35 μ1 of BB, were gently mixed with an equal volume of DNA at room temperature for 15 min. Following paramagnetic separation, the beads were washed using 35 μ1 of BB and resuspended in 15 μ1 of freshly prepared 0.1 M NaOH for 10 min. The solution was then magnetically separated from the beads and transferred into a second tube containing 7.5 μ1 of 0.2 M HC1. Then 1.88 μ1 of 1M TrisHC1 pH 8 was quickly added to the ssf probe solution. Finally, the ssf probe was further purified using the QIAquick PCR purification kit and eluted with 25 μ1 of EB.

### EXAMPLE 4

PCR-CRMS Assay.

All assays were carried out in 0.2 ml thin-walled test tubes. Solutions (10 μ1) containing 250 fmol of DNA corresponding to 3 μ1 of PCR product were first heat denatured and reannealed, in PCR-CRMS buffer, then pre-incubated at 37° C. until enzyme addition. The 10× PCR-CRMS buffer contained 200 mM Tris-HC1 pH 8, 100 μM EDTA, 7 mM DTT, 60 mM MgCl₂, 1 mg/ml BSA, 100 μM ADP, and 50-500 mM KC1. When the 260 bp product was used as target, 5% DMSO was added to the solution. The samples were heat denatured and reannealed, using the Robocycler™ (Stratagene), at 99° C. for 10 min immediately followed by a 15 min incubation at 60° C. The tubes were pre-incubated at 37° C. for 5-10 min. One to 1.5 μ1 of the purified His₆-MutS enzyme (260-390 ng, 275-410 nM) was first added for higher specificity (without ATP), for 20 min at 37° C. The endonuclease reaction was initiated by adding a cocktail of 1 μ1 His₆-MutH (25 ng, 85 nM), 1-1.5 μ1 His₆-MutL (120-180 ng, 180-270 nM) and 0.15 μ1 of ATP 100 mM (final 1.5 mM). The incubation was continued for 20 min at 37° C. The final KC1 concentration varied from 60-110 mM.

The self-reannealed reactions (mutation detection mode) were terminated using 10 μ1 of deionized formamide containing 25 mM EDTA and 0.05% bromophenol blue, then kept on ice until loaded on an 8M urea PAGE gel. Following electrophoresis, the gel was ™Vistra Green (Amersham LIFE SCIENCE) stained and scanned using a Fluorimager SI scanner (Vistra Fluorescence™). Fractions cleaved were quantified using the ImageQuaNT software.

### EXAMPLE 5

The fluorescence-based typing reactions (genotyping mode) were treated as above except that 10⁻²⁵ fmol of ssf probe was added to the solution prior to the denaturation/reannealing step. Reactions were stopped using 0.5 μ1 of 0.5 M EDTA, followed by a 30 min evaporation under low atmosphere. The resulting 2 μ1 solution was electrophoresed on a glycerol-tolerant 6% PAGE 8M urea gel containing 100 mM Tris-HC1, 28.75 mM taurine, and 500 μM EDTA at 45° C., 2500 V for 5 hr on the ABI PRISM™ 377 DNA Sequencer. The fraction digested was then quantitated from GeneScan™ electropherograms. For the analysis of the 5′UTR and part of the promoter region of the CDKN1A gene, a native gel system was used for better resolution of large DNA fragments (1257bp). The gel solution was the same as above, but lacking urea. Electrophoresis lasted for 12 hr at 1000 V and 30° C.

### EXAMPLE 6

Optimization of PCR-CRMS using the Taguchi Method.

A modified Taguchi method (Cobb et al., 1994; Lundberg et al. manuscript in preparation) was used to determine the optimal experimental conditions necessary to perform PCR-CRMS on different target DNA. Arrays of 4 parameters by 3 parameter values were used to measure the effects and interactions of specific reaction components simultaneously. The end point for PCR-CRMS optimization purpose, was the highest ratio between the fraction of endonuclease activity obtained from heterozygote and homozygote DNA samples. The optimal level for each component, by Taguchi analysis, was finally assayed, along with 2 or 3 slightly different conditions, to confirm the values as the most appropriate experimental conditions.

While the invention has been disclosed by reference to the details of preferred embodiments of the invention, it is to be understood that the disclosure is intended in an illustrative rather than a limiting sense, as it is contemplated that modifications will readily occur to those skilled in the art, within the spirit of the invention and the scope of the appended claims.

### REFERENCES

1. Halushka, M. K. et al. Patterns of single-nucleotide polymorphisms in candidate genes for blood-pressure homeostasis. Nat Genet 22:239-247 (1999).

2. Cargill, M. et al. Characterization of single-nucleotide polymorphisms in coding regions of human genes. Nat Genet 22:231-238 (1999).

3. Lambrinakos, A. et al. Reactivity of potassium permanganate and tetraethylammonium chloride with mismatched bases and a simple mutation detection protocol. Nucleic Acids Res 27:1866-1874 (1999).

4. Ellis, T. P. et al. Chemical cleavage of mismatch: a new look at an established method. Hum Mutat 11:345-53 (1998).

5. Cotton, R. G. et al. Reactivity of cytosine and thymine in single-base-pair mismatches with hydroxylamine and osmium tetroxide and its application to the study of mutations. Proc Natl Acad Sci U S A 85:4397-4401 (1988).

6. Myers, R. M. et al. Detection and localization of single base changes by denaturing gradient gel electrophoresis. Methods Enzymol 155:501-27 (1987).

7. Orita, M. et al. Detection of polymorphisms of human DNA by gel electrophoresis as single-strand conformation polymorphisms. Proc Natl Acad Sci U S A 86:2766-2770 (1989).

8. Liu, Q. et al. Detection of virtually all mutations- SSCP (DOVAM-S): a rapid method for mutation scanning with virtually 100% sensitivity. Biotechniques 26:932, 936-938, 940-942 (1999).

9. Heid, C. A. et al. Real time quantitative PCR. Genome Res 6:986-94 (1996).

10. Tyagi, S. et al. Multicolor molecular beacons for allele discrimination. Nat Biotechnol 16:49-53 (1998).

11. Kwiatkowski R. W. et al. Clinical, genetic, and pharmacogenetic applications of the Invader assay. Mol Diagn 4:353-364 (1999.

12. Ugozzoli, L. et al. Application of an allele-specific polymerase chain reaction to the direct determination of ABO blood group genotypes. Genomics 12:670-674 (1992)

13. Glavac, D. et al. Applications of heteroduplex analysis for mutation detection in disease genes. Hum Mutat 6:281-287 (1995).

14. Dianzani, I. et al. Fifth international mutation detection workshop, May 13-16, 1999, Vicoforte, Italy. Hum Mutat 14:451-453 (1999).

15. Cotton, R. G. Mutation detection by chemical cleavage. Genet Anal 14:165-168 (1999).

16. Taylor, G. R. et al. Enzymatic methods for mutation scanning. Genet Anal 14:181-186 (1999).

17. Nelson, S. F. et al. Genomic mismatch scanning: a new approach to genetic linkage mapping. Nat Genet 4:11-8 (1993).

18. McAllister, L. et al. Enrichment for loci identical-by-descent between pairs of mouse or human genomes by genomic mismatch scanning. Genomics 47:7-11 (1998).

19. Cheung, V. G. et al. Genomic mismatch scanning identifies human genomic DNA shared identical by descent. Genomics 47:1-6 (1998).

20. Cheung, V. G. et al. Linkage-disequilibrium mapping without genotyping. Nat Genet 18:225-230 (1998).

21. Nelson, S. F. Genomic mismatch scanning: current progress and potential applications. Electrophoresis 16:279-285 (1995).

22. Welford, S. M. et al. Detection of differentially expressed genes in primary tumor tissues using representational differences analysis coupled to microarray hybridization. Nucleic Acids Res 26:3059-3065 (1998).

23. Lahue, R. S. et al. DNA mismatch correction in a defined system. Science 245:160-164 (1989).

24. Parker, B. O. et al. Repair of DNA heteroduplexes containing small heterologous sequences in Escherichia coli. Proc Natl Acad Sci U S A 89:1730-1734 (1992).

25. Blackwell, L. J. et al. Nucleotide-promoted release of hMutSalpha from heteroduplex DNA is consistent with an ATP- dependent translocation mechanism. J Biol Chem 273:32055-32062 (1998).

26. Yamaguchi, M. et al. MutS and MutL activate DNA helicase II in a mismatch-dependent manner. J Biol Chem 273:9197-201 (1998).

27. Dao, V. et al. Mismatch-, MutS-, MutL-, and helicase II-dependent unwinding from the single-strand break of an incised heteroduplex. J Biol Chem 273:9202-7 (1998).

28. Cobb, B. D. et al. A simple procedure for optimising the polymerase chain reaction (PCR) using modified Taguchi methods. Nucleic Acids Res 22:3801-3805 (1994).

29. Smith, J , et al. Mutation detection with MutH, MutL, and MutS mismatch repair proteins. Proc Natl Acad Sci U S A 93:4374-9 (1996).

30. Lishanski, A. et al. Mutation detection by mismatch binding protein, MutS, in amplified DNA: application to the cystic fibrosis gene. Proc Natl Acad Sci U S A 91:2674-2678 (1994).

31. Eckert, K. A. et al. DNA polymerase fidelity and the polymerase chain reaction. PCR Methods Appl 1:17-24 (1991).

32. Tindall, K. R. et al. Fidelity of DNA synthesis by the Thermus aquaticus DNA polymerase. Biochemistry 27:6008-6013 (1988).

33. Barnes, W. M. PCR amplification of up to 35-kb DNA with high fidelity and high yield from lambda bacteriophage templates. Proc Natl Acad Sci U S A 91:2216-2220 (1994).

34. Blackwell, L. J. et al. DNA-dependent activation of the hMutSalpha ATPase. J Biol Chem 273:32049-54 (1998).

35. Krawczak, M. et al. Neighboring-nucleotide effects on the rates of germ-line single-base- pair substitution in human genes. Am J Hum Genet 63:474-88 (1998).

36. Hawkins, G. A. et al. Base excision sequence scanning. Nat Biotechnol 15:803-4 (1997).

37. Feng, G. et al. Single-step purifications of His6-MutH, His6-MutL and His6-MutS repair proteins of Escherichia coli K-12. Biotechniques 19:956-65 (1995).

38. Law, J. C. et al. Identification of a PstI polymorphism in the p21Cipl/Wafl cyclin-dependent kinase inhibitor gene. Hum Genet 95:459-460 (1995).

39. Ban, C. and Yang, W. Crystal structure and ATPase activity of MutL: implications for DNA repair and mutagenesis. Cell., 95:541-552 (1998).

40. Young, M. C., Kuhl, S. B. and von Hippel, P. H. Kinetic theory of ATP-driven translocases on one-dimensional polymer lattices. J Mol Biol., 235:1436-1446.

41. Young, M. C. Schultz, D. E., Ring, D. And von Hippel, P. H. Kinetic parameters of the translocation of bacteriophage T4 gene 41 protein helicase on single-stranded DNA. J Mol Biol., 235:1447-1458.

42. Gradia, S., Acharya, S. And Fishel, R. The human mismatch recognition complex hMSH2-hMSH6 functions as a novel molecular switch. Cell, 91:995-1005.

43. Hall, M. C. and Matson, S. W. The Escherichia coli MutL protein physically interacts with MutH and stimulates the MutH-associated endonuclease activity. J. Biol Chem., 274:1306-1312.

1. A method of genotyping or detecting a mutation in a DNA sample by candidate region mismatch scanning, which comprises:
(a) amplifying a candidate region of the DNA that includes at least one 5′ GATC 3′ site,
(b) denaturing and reannealing the amplified DNA,
(c) digesting the reannealed DNA with the E. coli mismatch detection enzymes, MutS, MutL and MutH, to cleave mismatch-containing DNA at the 5′ GATC 3′ site, and
(d) determining the fraction of DNA cleaved.

2. Method of claim 1 wherein step (a) is carried out by polymerase chain reaction using a high-fidelity DNA polymerase.

3. Method of claim 2 wherein step (b) is carried out in an assay buffer at about 99° C. for about 10 minutes and about 60° C. for about 15 minutes, the buffer containing (i) about 85 mM potassium chloride when the candidate region is about 260 or fewer bp and proportionately higher concentration of potassium chloride when the candidate region is more than about 260 bp; and (ii) about 100 μM ADP and about 5% DMSO when the candidate region is about 260 or fewer bp, less or no ADP and DMSO when the candidate region is more than about 260 bp, and no ADP or DMSO when the candidate region is about 516 or more bp.

4. Method of claim 3 wherein step (c) is carried out in the assay buffer containing about 25nM DNA, about 85 nM MutH, about 275nM MutS, about 1.5 mM ATP, about 180 nM MutL when the candidate region is about 260 or fewer bp, and proportionately higher concentration of MutL when the candidate region is more than about 260 bp.

5. Method of claim 4 wherein the 5′ GATC 3′ site is at least about 45 bp from the end of the candidate region and wherein the mismatch and 5′ GATC 3′ sites are separated by up to about 1 kb.

6. Method of claim 5 wherein the DNA polymerase used for PCR is a mixture of thermostable Taq and Pwo polymerases.

7. Method of claim 6 wherein step (d) is carried out by electrophoresing the digested DNA and quantitating the resulting bands.

8. Method of claim 7 wherein step (a) is carried out with a radioactive- or fluorescent-labeled primer.

9. Method of claim 6 wherein step (a) is carried out with mass-tagged primers and step (d) is carried out by mass spectroscopy.

10. Method of claim 6 wherein step (d) is carried out by mass spectroscopy.

11. Method of claim 1 wherein the candidate region contains more than one polymorphism.

12. A method of genotyping a DNA sample by candidate region mismatch scanning, which comprises:
(a) amplifying a candidate region of the DNA that includes at least one 5′ GATC 3′ site, and mixing the amplified DNA with a detectably-labeled probe prepared by amplifying the corresponding region of a homozygous reference sample,
(b) denaturing the amplified DNA and reannealing in presence of the probe to produce unlabeled homoduplexes and labeled heteroduplexes,
(c) digesting the reannealed DNA with the E. coli mismatch detection enzymes, MutS, MutL and MutH, to cleave mismatch-containing DNA at the 5′ GATC 3′ site; and
(d) determining the fraction digested of the single stranded labeled probe.

13. Method of claim 12 wherein step (a) is carried out by polymerase chain reaction using a high-fidelity DNA polymerase.

14. Method of claim 13 wherein step (b) is carried out in an assay buffer at about 99° C. for about 10 minutes and about 60° C. for about 15 minutes, the buffer containing (i) about 60 mM potassium chloride when the candidate region is about 260 or fewer bp and proportionately higher concentration of potassium chloride when the candidate region is more than about 260 bp; and (ii) about 100 μM ADP and about 5% DMSO when the candidate region is about 260 or fewer bp, less or no ADP and DMSO when the candidate region is more than about 260 bp, and no ADP or DMSO when the candidate region is about 516 or more bp.

15. Method of claim 14 wherein step (c) is carried out in the assay buffer containing about 25nM DNA, about 85 nM MutH, about 275nM MutS, about 1.5 mM ATP, about 180 nM MutL when the candidate region is about 260 or fewer bp, and proportionately higher concentration of MutL when the candidate region is more than about 260 bp.

16. Method of claim 15 wherein the 5′ GATC 3′ site is at least about 45 bp from the end of the candidate region and the mismathch and 5′ GATC 31 sites are separated by up to about 1 kb.

17. Method of claim 16 wherein the DNA polymerase used for PCR is a mixture of thermostable Taq and Pwo polymerases.

18. Method of claim 17 wherein the probe is labeled with a fluorescent or radioactive label or a mass tag.

19. Method of claim 18 wherein the probe is a single-stranded probe that is labeled at one end, a double-stranded probe that is labeled at one end of one strand, or a double-stranded probe that is labeled at one end of one strand with one label and at one end of the opposite strand with a different label.

20. Method of claim 19 wherein the label or each label is fluorescent and wherein step (d) is carried out by electrophoresing the digested DNA on an automated DNA sequencer and the fraction digested is quantitated from electropherograms.

21. Method of claim 20 wherein the reaction steps (a), (b) and (c) are carried out in multiple well plates under inert mineral oil to prevent evaporation.

22. Method of claim 21 wherein the probe is mass-tagged and step (d) is carried out by mass spectroscopy.

23. Method of claim 12 wherein the candidate region contains more than one polymorphism.

24. A method of determining allele-sharing status between sibs by candidate region mismatch scanning which comprises
(a) separately amplifying corresponding candidate regions of genomic DNA samples from a sib pair, which candidate regions contain at least one 5′ GATC 3′ site, labeling one amplified DNA with a detectable label, and mixing the unlabeled and labeled amplified DNA's, with the unlabeled DNA present in sufficient excess to maintain the quantitative aspects of the method,
(b) denaturing the mixed amplified DNA's and reannealing to produce labeled homoduplexes and labeled heteroduplexes,
(c) digesting the reannealed DNA with the E. coli mismatch detection enzymes, MutS, MutL, and MutH, to cleave mismatch-containing DNA at the 5′ GATC 3 site, and
(d) determining the fraction of the labeled DNA cleaved.

25. Method of claim 24 wherein step (a) is carried out by polymerase chain reaction using a high-fidelity DNA polymerase.

26. Method of claim 25 wherein step (b) is carried out in an assay buffer at about 99° C. for about 10 minutes and about 60° C. for about 15 minutes, the buffer containing (i) about 60 mM potassium chloride when the candidate region is about 260 or fewer bp and proportionately higher concentration of potassium chloride when the candidate region is more than about 260 bp; and (ii) about 100 μM ADP and about 5% DMSO when the candidate region is about 260 or fewer bp, less or no ADP and DMSO when the candidate region is more than about 260 bp, and no ADP or DMSO when the candidate region is about 516 or more bp.

27. Method of claim 26 wherein step (c) is carried out in the assay buffer containing about 25nM DNA, about 85 nM MutH, about 275nM MutS, about 1.5 mM ATP, about 180 nM MutL when the candidate region is about 260 or fewer bp, and proportionately higher concentration of MutL when the candidate region is more than about 260 bp.

28. Method of claim 27 wherein the 5′ GATC 3′ site is at least about 45 bp from the end of the candidate region and the mismatch and 5′ GATC 3′ sites are separated by up to about 1 kb.

29. Method of claim 28 wherein the DNA polymerase used for PCR is a mixture of thermostable Taq and Pwo polymerases.

30. Method of claim 29 wherein the labeled amplified DNA is labeled during amplification by use of a labeled primer or a pair of labeled primers.

31. Method of claim 30 wherein the labeled amplified DNA is labeled during amplification by use of a 5′-fluorescent labeled primer or a pair of 5′-fluorescent labeled primers.

32. Method of claim 31 wherein the labeled DNA is mixed with unlabeled DNA in a ratio in the range of about 1:5 to about 1:30.

33. Method of claim 24 wherein the candidate region contains more than one polymorhism.

34. Method of claim 1 wherein multiple candidate regions are mismatch scanned simultaneously in the same container by amplifying candidate regions of different sizes that produce cleavage products of different sizes or by amplifying different candidate regions with different labels.
